(19) United States
(12) Patent Application Publication
(10) Pub. No.: US 2007/0071808 A1
(43) Pub. Date: Mar. 29, 2007

(54) Capsule filled with honey

(76) Inventors: John J. Janik, Kensington, CT (US); Susan B. Janik, Berlin, CT (US); Dorothy L. Carlone, Berlin, CT (US)

Correspondence Address: MCCARTER & ENGLISH LLP;CITYPLACE I, 185 ASYLUM STREET, HARTFORD, CT, 06103, US

(21) Appl. No.: 11/605,587

(22) Filed: Nov. 29, 2006

Related U.S. Application Data
(63) Continuation-in-part of application No. 10/650,912, filed on Aug. 28, 2003.

(51) Int. Cl. A61K 9/48; A61K 35/64

(52) U.S. Cl. 424/451; 424/539

(57) ABSTRACT

Capsules containing honey or other sweeteners that may be added to beverages are described. The capsules may be made from a material that dissolves in water or other fluids, or, alternatively, the capsule may be remain intact in a fluid and provide an edible capsule sweetener. The capsule may contain nutritional supplements or other additives in addition to the sweetener. Methods of using the capsules to sweeten beverages are also provided.

### RELATED APPLICATIONS

This application is a continuation-in-part of U.S. application Ser. No. 10/650,912 filed on Aug. 28, 2003.

### FIELD OF THE INVENTION

The present invention is directed to a capsule containing honey or other sweeteners that may be added to beverages. The capsule may be made from a material that dissolves in water or other fluids, or, alternatively, the capsule may be remain intact in a fluid and provide an edible capsule sweetener. The capsule may contain nutritional supplements or other additives in addition to the sweetener.

### BACKGROUND OF THE INVENTION

Beverages such as tea, coffee or other liquids often require sweetening prior to consumption to make the beverage more palatable to an individual. Among the sweeteners used for this purpose are honey, sugar or artificial sweeteners. These sweeteners can be messy and difficult to use, particularly in a work place. The sweeteners can be sticky when spilled, can be difficult to handle, and if not properly cleaned can attract insects.

In addition to hot beverages, cold beverages may require sweetening, or it may be desirable to provide an edible capsule containing a sweetener or other food product. For example, in one type of cold beverage known as bubble tea, a beverage generally made with tea, sugar, milk and black tapioca pearls. The tea, milk and sugar are shaken to form a foam, which gives the drink its name. The tapioca pearls are made from tapioca starch obtained from tapioca or the bitter-casava plant. A consumer drinks the liquid, and can chew on the tapioca pearls.

Accordingly, it would be desirable to have a food product for use with beverages, preferably tea or other hot beverages, that can provide sweetening to the beverage. The food product should be easy to use and to handle. When used to sweeten a beverage, the food product should readily dissolve in fluids, particularly in hot water.

In another embodiment, the food product is made to be edible, but does not readily dissolve in a cold fluid, such as for example bubble tea, to provide a capsulized food product that can be chewed or eaten.

### SUMMARY OF THE INVENTION

The present invention is directed to a capsule filled with honey or another sweetener. The capsule is comprised of a material, such as for example hydroxymethyl cellulose. In one embodiment, the capsule is soluble in water, particularly in warm or hot water. This embodiment of the invention may be used to sweeten a beverage for drinking. The capsule may be in any size and shape that is appropriate to provide the desired amount of a sweetener for addition to a beverage, such as tea or other hot beverages.

In another embodiment, the capsule is edible, but does not readily dissolve in a beverage that it at room temperature or below. This embodiment may be used to provide a chewable product that may be either chewed directly, or may be used in a cold beverage such as bubble tea. In this embodiment, the capsule is preferably sized and shaped to allow the capsule to be readily sucked through a straw when drinking the beverage.

In addition to honey or other sweeteners, the capsule may contain other nutritional supplements, such as vitamins or minerals, to provide a fortified beverage. For example, Vitamins A, D, C, or E may be included, or minerals such as calcium may be added to the capsule.

Among the advantages of the present invention is that the sweetener is easy to use and to handle. The capsules may be provided in a bottle or other container, and they may be added to a beverage without the need for utensils. Because the honey or other sweetener is contained within the capsule, there is less likelihood that the sweetener will spill.

Other advantages of the invention will be apparent in view of the detailed description of preferred embodiments provided below.

### BRIEF DESCRIPTION OF THE DRAWINGS

FIG. 1 is a view of a capsule containing honey or other sweetener.

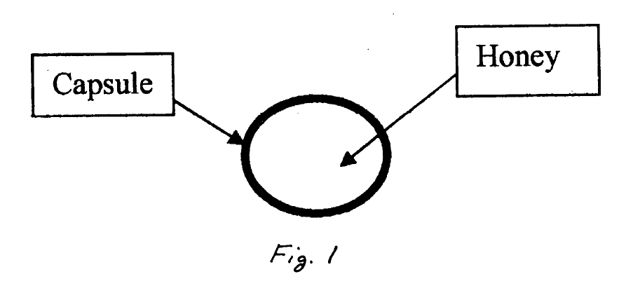

### DETAILED DESCRIPTION OF THE INVENTION

The present invention, sometimes referred to herein as “Drops of Honey” is a food product composition that may be used to sweeten beverages or that may be used as a chewable food product. Drops of Honey is a capsule filled with honey or other sweetener. In one embodiment, the product can be used to sweeten with tea, coffee or other hot beverages. In this embodiment, the capsule dissolves in water, particularly in hot or boiling water, and allows the honey or other sweetener to escape into the beverage of choice. Preferably, substantially all of the capsule dissolves without leaving any noticeable residue in the beverage.

In another embodiment, the product can be used as a chewable product that can either be chewed directly, or that may be added to a cold beverage such as bubble tea. In this embodiment, the capsule is made such that the capsule does not readily dissolve in a fluid at room temperature or below. The capsule may be chewed to release the sweetener within the capsule.

The capsule may be composed of any appropriate material that may be consumed by humans. The capsule material and the thickness of the capsule is selected depending upon the desired use of the capsule, i.e. for dissolution in the beverage or for use as a chewable food product at room temperature or below, and based upon the amount of honey or other sweetener that will be contained in the capsule. The material, size and thickness of the capsule can be selected to achieve a desired dissolution rate, particularly in hot fluids, or to provide a chewable capsule for use, for example, in bubble tea. In a preferred embodiment, hydroxymethylcellulose (“HMC”) is used as the material for the capsule. In this embodiment, the capsule preferably comprises about 92% HMC and about 8% water. In other embodiments of the invention, and a gelatin capsule or a vegetable based capsule may be used.

Gelatin capsules typically include animal products, such as for example beef gelatin or pork gelatin, and can be more cost efficient to produce. The capsules may include other ingredients as appropriate, including glycerine or water.

Vegetable based capsules do not contain animal by-products, and may be preferred by individuals who wish to avoid animal products in foodstuffs. Vegetable based capsules come in various forms, and they are typically made from materials derived from vegetable cellulose. An example of a vegetable based capsules include Vegicaps® Soft capsules manufactured by Cardinal Health, which are composed of seaweed extract and gluten free starch. The invention is not limited in this regard, and any other vegetable based capsule that will dissolve in warm or hot water may be used.

The capsule may be any size or shape that is appropriate to contain the desired amount of honey or other sweetener, that can be conveniently packaged in bottles or other appropriate containers, and can be readily handled by the user. In preferred embodiments, the capsules are oblong, oval or spherical. The diameter of the capsule is preferably between ¼ inch and ¾ inch. For a capsule that is not spherical, the length of the capsule is preferably between ¼ inch and ¾ inch. In a particularly preferred embodiment for use in warm beverages, the capsule is in the form of a ball that is approximately ⅜ inches in diameter. For use in cold beverages, the capsule is preferably sized to readily pass through a straw when sucked by a consumer of a cold beverage.

The capsules are filled with a sweetener, which is preferably provided in a liquid formulation for ready mixing with the beverage after the capsule has dissolved or for eating if the capsule is chewed. For example, the capsule may contain sugar, preferably in the form of a solution that provides the desired amount of sugar for sweetening a beverage. In a preferred embodiment, the capsule contains honey, which can provide natural sweetening to the beverage or when chewed, as well as providing other nutritional value. In this embodiment, the capsule preferably contains between about 400 i.u. and about 600 i.u. of honey if used to dissolve in a hot beverage, and preferably between about 600 i.u and about 800 i.u. of honey if used as a chewable capsule in, for example, bubble tea.

In one embodiment of the invention, the capsule contains honey as the only filler. In other embodiments, in addition to honey or sweetener, the capsule may contain vitamins, minerals or other nutritional supplements, such as for example vitamins A, D, E or F or calcium. Including additional nutritional supplements in the capsule will allow the user to fortify tea or other beverage with desired nutrients. Preferably, the vitamin, mineral or other supplement is provided in a liquid form to avoid any residue in the beverage.

The following examples are provided for illustrative purposes only, and they are not intended to limit the present invention in any way.

### EXAMPLE 1

A gelatin capsule that contained 400 iu of honey was placed in an eight ounce cup of water at 75° C. with stirring for approximately 7 seconds. At 21 seconds, minute residue was seen in the water, but at 45 seconds the residue had dissolved.

### EXAMPLE 2

A vegetable based capsule that contained 400 iu of honey was placed in an eight ounce cup of water at 75° C. with stirring for approximately 7 seconds. Minute residue was observed at 22 seconds, which was completely dissolved at 46 seconds.

### EXAMPLE 3

Eight capsules of the type described in Examples 1 and 2 above were added to an eight ounce cup of water at 75° C. with stirring. As described above, residue was observed, but eventually dissolved.

### EXAMPLE 4

Four capsules of the type described in Examples 1 and 2 above were added to an eight ounce cup of water at 50° C. without stirring. The capsules were 80% dissolved in 10 minutes, and completely dissolved by 15 minutes.

### EXAMPLE 5

Four capsules of the type described in Examples 1 and 2 above were added to an eight ounce cup of water at 75° C. without stirring. The capsules were 80% dissolved in 5 minutes, and completely dissolved by 10 minutes.

What is claimed is:

1. A capsule for adding a sweetener or other additive to a beverage comprising:
a. a hydroxymethyl cellulose capsule; and
b. at least one water soluble additive contained within the hydroxymethyl cellulose capsule.

2. The capsule of claim 1, wherein the hydroxymethyl cellulose capsules comprise about 92% hydroxymethyl cellulose and about 8% water.

3. The capsule of claim 1, wherein the water soluble additive is a sweetening agent.

4. The capsule of claim 3, wherein the sweetening agent is honey.

5. The capsule of claim 4, wherein the capsule contains between about 400 i.u. and about 800 i.u. of honey.

6. The capsule of claim 5, wherein the capsule is substantially sphereical with a diameter of about ¾ inches.

7. The capsule of claim 3, wherein the capsule further contains a nutritional supplement.

8. The capsule of claim 6, wherein the nutritional supplement is selected from the group consisting of Vitamin A, D, E, F or calcium.

9. A method for adding a sweetener or other additive to a beverage comprising the steps of:
a. providing a beverage in a container;
b. adding to the beverage at least one capsule comprising
(i) a hydroxymethyl cellulose shell; and
(ii) at least one water soluble additive; and
c. agitating the beverage to dissolve the hydroxymethyl cellulose shell and water soluble additive in the beverage.

10. The method of claim 8, wherein the hydroxymethyl cellulose capsules comprise about 92% hydroxymethyl cellulose and about 8% water.

11. The method of claim 8, wherein the water soluble additive is a sweetening agent.

12. The method of claim 10, wherein the sweetening agent is honey.

13. The method of claim 11, wherein the capsule contains between about 400 i.u. and about 800 i.u. of honey.

14. The method of claim 13, wherein the capsule is substantially sphereical with a diameter of about ¾ inches.

15. The method of claim 10, wherein the capsule further contains a nutritional supplement.

16. The method of claim 13, wherein the nutritional supplement is selected from the group consisting of Vitamin A, D, E, F or calcium.
